(19) United States
(12) Patent Application Publication
(10) Pub. No.: US 2012/0028929 A1
(43) Pub. Date: Feb. 2, 2012

(54) TETRACYCLINE STABILIZING FORMULATIONS

(75) Inventors: David F. Power, Trabuco Canyon, CA (US); Greg Fieldson, Morgantown, WV (US); Yunik Chang, Sonoma, CA (US)

(73) Assignee: ALACRITY BIOSCIENCE, INC., Laguna Hills, CA (US)

(21) Appl. No.: 13/119,547

(22) PCT Filed: Sep. 18, 2009
(86) PCT No.: PCT/US09/57475

§ 371 Date: Oct. 21, 2011

Related U.S. Application Data
(60) Provisional application No. 61/192,522, filed on Sep. 19, 2008.

(51) Int. Cl. A61K 31/65; A61P 31/04; A61P 27/02; A61P 17/00; A61P 29/00; A61P 17/10

(52) U.S. Cl. 514/153

(57) ABSTRACT

An aqueous solution comprising a chelating agent at a concentration of about 0.1-0.5%, and an antioxidant agent at a concentration of about 0.1-0.5%, wherein the pH of the solution is between about 4.5 and about 7.5 is disclosed. The aqueous solution may comprise a tetracycline composition wherein the tetracycline is formulated in the aqueous solution. A method for reducing the rate and/or overall extent of degradation of a tetracycline in aqueous solution, comprising admixing in a tetracycline containing solution a chelating agent at a concentration of about 0.1-0.5% and an antioxidant at a concentration of about 0.1-0.5% and, as necessary, adjusting the pH of the solution so that it is between about 4.5 and about 7.5 is also disclosed. Tetracycline compositions of the invention may be used to treat inflammatory and/or tissue degeneration conditions.

### BACKGROUND OF THE INVENTION

Tetracyclines are a group of broad-spectrum antibiotics used in a variety of indications. Tetracyclines, as an antibiotic, are used to treat a wide range of bacterial infections, for example pneumonia and other respiratory tract infections, acne, infections of skin, genital and urinary systems (e.g., prostatitis, sinusitis, syphilis, chlamydia, and pelvic inflammatory disease), and a bacterial infection that causes stomach ulcers (Helicobacter pylori). Tetracyclines may also be used as an alternative or in addition to other medications for the treatment of various other diseases, such as Lyme disease and anthrax, and for the prevention of, for example, anthrax and malaria.

In addition to their antimicrobial activities, certain antibiotic compounds, such as tetracyclines, are known to exhibit anti-inflammatory actions. For example, metronidazole and ciprofloxacin both have anti-inflammatory and immuno-suppressant effects that may be more important than their antimicrobial effects. Tetracyclines and minocycline have been used to treat rheumatoid arthritis. Gentamycin, an aminoglycoside antibiotic, may exhibit an anti-inflammatory action due to inhibition of neutrophil NADPH oxidase activation. The quinolones show anti-inflammatory activity by reducing the potent reactive oxygen species excessively generated by neutrophils at the sites of inflammation. This leads to a reduction in oxidative tissue injury. Antibiotics are also known to have anti-proteolytic properties. Tetracyclines act as anti-inflammatory and anti-proteolytic agents through a number of different pathways. Doxycycline, for example, inhibits phorbol-12-myristate-13-acetate-mediated matrix metalloproteinase 8 (MMP-8) and MMP-9. Doxycycline also decreases elastin degradation, modulates nitric oxide synthesis, reduces MMP activity and inhibits the production of IL-1β. As such, tetracyclines are also thought to be useful in treating various inflammatory conditions, such as, e.g., acne vulgaris, rosacea, bullous dermatoses, rheumatoid arthritis, granulomatous disease, livedo vasculitis, sterile corneal ulceration and periodontitis.

Tetracyclines and tetracycline analogs are unstable in aqueous solution. This instability can lead to decreased activity of the compound over time and to a short shelf life of pharmaceutical formulations containing a tetracycline. For example, doxcycline in aqueous solution at neutral pH has a half-life of about 2 weeks at room temperature. This problem is typically addressed by storing tetracyclines in other forms, such as in lyophilized powders. However, such forms must be prepared in aqueous solution before administering the compound to the patient. Moreover, reconstituted parenteral solutions of tetracyclines are only considered usable for 6-48 hours, depending on the specific saline formulation (FDA). Thus, such compositions are inconvenient, costly, and susceptible to human error. Accordingly, formulations that allow for greater tetracycline compound stability in aqueous solution, which are also safe for ingestion, injection, and/or topical application for human subjects, would greatly increase the usability and decrease the cost of this important treatment option.

### SUMMARY OF THE INVENTION

In one aspect, the present invention provides a tetracycline composition comprising a tetracycline formulated in an aqueous solution comprising a chelating agent at a concentration of about 0.1-0.5%, and an antioxidant agent at a concentration of about 0.1-0.5%, wherein the pH of the solution is between about 4.5 and about 7.5. In another aspect, the invention provides an aqueous solution comprising a chelating agent at a concentration of about 0.1-0.5%, and an antioxidant agent at a concentration of about 0.1-0.5%, wherein the pH of the solution is between about 4.5 and about 7.5.

In certain embodiments, the chelating agent is disodium edetate. An antioxidant agent according to the invention includes, without limitation: sodium bisulfite, sodium metabisulfite, sodium thiosulfate, and thiourea. In certain embodiments, the antioxidant agent is a combination of sodium metabisulfite and sodium thiosulfate. Other antioxidant agents may be used, alone or in various combinations.

In certain embodiments, the composition further comprises a buffering agent. In a further embodiment, the buffering agent comprises sodium phosphate dibasic and citric acid, wherein the concentration of sodium phosphate dibasic is from about 0.05% to about 0.2% and the concentration of citric acid is from about 0.025% to about 0.1%.

In certain embodiments, the composition further comprises a tonicity modifier. In a further embodiment, the tonicity modifier is glycerin, wherein the concentration of glycerin is from about 0.5% to about 2%.

In certain embodiments, the pH of the composition is between about 5.2 and about 5.8.

In certain embodiments, the tetracycline is in the monohydrate or hyclate form. In certain embodiments, the tetracycline is a tetracycline analog. In yet other embodiments, the tetracycline is a chemically modified tetracycline (CMT). The tetracycline analog according to the invention includes, without limitation: oxytetracycline, doxycycline and minocycline. In certain embodiments, the tetracycline analog is doxycycline at a concentration from about 0.0025% to about 1%.

In any of the above described embodiments, the composition may be further characterized as a pharmaceutical composition and further comprises a pharmaceutically acceptable carrier.

In another aspect, the present invention provides a method for reducing the rate and/or overall extent of degradation of a tetracycline in aqueous solution, comprising admixing in a tetracycline containing solution a chelating agent at a concentration of about 0.1-0.5% and an antioxidant at a concentration of about 0.1-0.5% and, as necessary, adjusting the pH of the solution so that it is between about 4.5 and about 7.5.

In certain embodiments, the chelating agent is disodium edetate. The antioxidant agent according to the invention includes, without limitation: sodium bisulfite, sodium metabisulfite, sodium thiosulfate and thiourea. In certain embodiments, the antioxidant agent is a combination of sodium metabisulfite and sodium thiosulfate. Other antioxidant agents may be used, alone or in various combinations.

In certain embodiments, the method further comprises a step of admixing in the tetracycline solution a buffering agent. In further embodiments, the buffering agent comprises sodium phosphate dibasic and citric acid, wherein the concentration of sodium phosphate dibasic is from about 0.05% to about 0.2% and the concentration of citric acid is from about 0.025% to about 0.1%.

In certain embodiments, the method further comprises the step of admixing in the tetracycline solution a tonicity modifier. In further embodiments, the tonicity modifier is glycerin at a concentration of from about 0.5% to about 2%.

In certain embodiments, the pH of the aqueous solution is adjusted so that it is between about 5.2 and about 5.8.

In certain embodiments, the tetracycline is in the monohydrate or hyclate form. In certain embodiments, the tetracycline is a tetracycline analog. In yet other embodiments, the tetracycline is a chemically modified tetracycline (CMT). A tetracycline analog according to the invention includes, without limitation: oxytetracycline, doxycycline and minocycline. In certain embodiments, the tetracycline analog is doxycycline at a concentration of from about 0.0025% to about 1%.

Another aspect of the invention provides a method for treating a patient suffering from a condition associated with inflammation and/or tissue degeneration, said method comprising administering to a patient in need thereof a composition comprising an effective amount of a tetracycline in an aqueous solution, the solution further comprising a chelating agent at a concentration of about 0.1-0.5%, and an antioxidant agent at a concentration of about 0.1-0.5%, wherein the pH of the solution is between about 4.5 and about 7.5.

In certain embodiments, the chelating agent is disodium edetate. An antioxidant agent according to the invention includes, without limitation: sodium bisulfite, sodium metabisulfite, sodium thiosulfate and thiourea. In certain embodiments, the antioxidant agent is a combination of sodium metabisulfite and sodium thiosulfate. Other antioxidant agents may be used, alone or in various combinations.

In certain embodiments, the composition comprises a buffering agent. In further embodiments, the buffering agent is a phosphate citrate buffer wherein the concentration of sodium phosphate dibasic is from about 0.05% to about 0.2% and the concentration of citric acid is from about 0.025% to about 0.1%. In certain embodiments, the composition comprises a tonicity modifier. In a further embodiment, the tonicity modifier is glycerin at a concentration of from about 0.5% to about 2%.

In certain embodiments, the pH of the composition is between about 5.2 and about 5.8.

In certain embodiments, the tetracycline is in the monohydrate or hyclate form. In certain embodiments, the tetracycline is a tetracycline analog. In yet other embodiments, the tetracycline is a chemically modified tetracycline (CMT). The tetracycline analog according to the invention includes, without limitation: oxytetracycline, doxycycline and minocycline. In certain embodiments, the tetracycline analog is doxycycline at a concentration of from about 0.0025% to about 1%.

A condition associated with inflammation or tissue degeneration which may be treated according to the invention includes, without limitation: acne vulgaris, rosacea, bullous dermatoses, rheumatoid arthritis, granulomatous disease, livedo vasculitis, sterile corneal ulceration, dry eye disease, macular degeneration, recurrent corneal corrosion and diabetic retinopathy.

### BRIEF DESCRIPTION OF THE DRAWINGS

FIG. 1 shows the level of apical corneal desquamation due to 5 days of Experimental Dry Eye (EDE) in mice treated with topical application of the following formulations: DOXY (doxycycline 0.025% w/w); DOXY 10 (doxycycline 0.0025%); EPI6 (6-epidoxycyline 0.025%); EPI4 (4-epidoxycyline 0.025%); DOXY+EPI4 (doxycycline 0.0125% and 4-epidoxycyline 0.0125%); DOXY+EPI6 (doxycycline 0.0125% and 6-epidoxycyline 0.0125%); VEHICLE (formulation vehicle); 5D (5 days of EDE w/o treatment); UT (untreated control—no EDE, no treatment). FIG. 1A shows the percent loss of apical epithelial cells coverage, calculated as the area of desquamation in relation to the photographic field. FIG. 1B shows the mean apical epithelial cell area, measured by drawing out and calculating the mean surface area of 8-9 individual cells. FIG. 1C shows the cell density in the apical epithelium and was calculated by dividing the photographic field with the mean cell area and normalizing to square millimeters (mm²). FIG. 1D shows the number of cells lost to desquamation, and was calculated by dividing the area of desquamation by the mean cell area.

### DETAILED DESCRIPTION OF THE INVENTION

In order that the invention herein described may be fully understood, the following detailed description is set forth. Various embodiments of the invention are described in detail and are further illustrated by the provided, non-limiting examples.

Unless defined otherwise, all technical and scientific terms used herein have the same meaning as those commonly understood by one of ordinary skill in the art to which this invention belongs. Although methods and materials similar or equivalent to those described herein may be used in the invention or testing of the present invention, exemplary suitable methods and materials are described below. The materials, methods and examples are illustrative only, and are not intended to be limiting.

All publications, patents, patent publications and applications and other documents mentioned herein are incorporated by reference in their entirety.

In order to further define the invention, the following terms and definitions are provided herein.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Throughout this specification, the word “comprise” or variations such as “comprises” or “comprising” will be understood to imply the inclusion of a stated integer or groups of integers but not the exclusion of any other integer or group of integers.

As used herein, tetracycline and its analogs which have antimicrobial activity at physiologically useful concentrations are termed “antimicrobial tetracyclines.” Antimicrobial tetracyclines may, alternatively, be administered at concentrations that are sub- or non-antimicrobial.

As used herein, chemically modified tetracycline analogs or “chemically modified tetracyclines (CMTs)” may or may not lack antimicrobial activities at certain or all concentrations. Those that lack antimicrobial activity at any concentration are referred to herein as “non-antimicrobial tetracyclines.”

Unless otherwise specified, the term “tetracycline” is used herein to refer generically to tetracyclines, tetracycline analogs, and CMTs.

As used herein, the term “chelating agent” refers to an agent that is capable of bonding or forming a complex with one or more metal ions. For example, a chelating agent of the invention includes, without limitation, disodium edetate, ethylenediamine tetraacetic acid (EDTA), dimercaprol, diethylenetriaminepentaacetic acid (DTPA), N (hydroxyethyl) ethylenediaminetriacetic acid (HEDTA), nitrilotriacetic acid (NTA), or an agent based on any of these chelating agents.

As used herein, the term “anti-oxidant” refers to a molecule that is capable of slowing or preventing the oxidation of other molecules. For example, anti-oxidant agents which may be used in the invention include, without limitation, sodium metabisulfite, sodium thiosulfate, sodium bisulfite, thiourea or an agent based on any of these anti-oxidant agents.

The present invention provides compositions, including pharmaceutical compositions, comprising a compound formulated to have increased stability in aqueous solution compared to that of the same compound formulated in a standard balanced salt solution. In certain embodiments, the compounds are formulated in an aqueous solution comprising a chelating agent and an antioxidant. Optionally, the pH of the solution is adjusted to between 4.5 to 7.5 or about 5.5. This pH adjustment may be empirically determined depending on the particular compound to be stabilized. In certain embodiments, the compound is a tetracycline. In other embodiments, the compound is a derivative or analog of a tetracycline, or a chemically modified tetracycline (CMT). The present invention also provides methods for decreasing the rate and/or overall amount of degradation of a tetracycline in aqueous solution. The present invention also provides methods for treating a patient in need thereof by administering a stabilized tetracycline aqueous solution of the invention, alone or in combination with one or more additional therapeutic agents or treatment regimens.

Tetracycline will degrade into epimers or oxidative degradation products when stored in aqueous solution, thus decreasing the shelf life of the compound. For example, the tetracycline derivative doxycycline will degrade into 4-epidoxycycline and 6-epidoxycycline and other degradation products when in solution. The present invention provides compositions and methods for reducing and/or slowing down the degradation of tetracycline and its derivatives or analogs into degradation products or epimers when in aqueous solution.

The stability of a tetracycline in solution can be defined as the length of time the tetracycline remains at or above a certain minimum concentration without degrading into its epimers or degradation products. In certain embodiments, the stability of tetracycline in aqueous solution will be such that, when stored at about 5° C., 75% to about 80%, about 80% to about 85%, about 85% to about 90%, about 90% to about 95%, or about 95% to about 99% or higher of the tetracycline remains in solution after a period of about 12 months to about 15 months, about 15 months to about 18 months, about 18 months to about 21 month, about 21 months to about 24 months, about 24 months to about 27 months, about 27 months to about 30 months, about 30 months to about 36 months, or about 36 months to about 42 months, or more than 42 months.

In certain embodiments, the present invention may be used to stabilize tetracycline in aqueous solution so that more than 80% of the total tetracycline compound remains after about 18 months to about 24 months, about 24 months to about 30 months, or after more than 30 months. In other embodiments, the present invention is used to stabilize tetracycline in aqueous solution so that more than 90% of the total tetracycline compound remains after about 18 to about 24 months, about 24 to about 30 months, or after more than 30 months.

Standard methods known in the art are used for monitoring the degradation of tetracycline into its epimers or degradation products. For example, one method for monitoring tetracycline degradation is an analytical technique using chromatography, e.g. high pressure liquid chromatography (HPLC), to separate the various degradants, e.g. epimers and other degradants, and a sensitive detector to identify and quantify the concentrations of the drug and degradants over time.

In certain embodiments, the present invention provides methods for slowing or decreasing the overall level of degradation of tetracycline in aqueous solution. Such a method comprises the step of admixing in the solution a chelating agent and an antioxidant, as described in detail above. In certain embodiments, the method further comprises the step of adjusting the pH of the solution, as described in detail above. In certain embodiments, the method further comprises the step of adjusting the storage temperature of the solution. In certain specific embodiments, the method comprises the steps of admixing in the solution 0.2% (w/w) edetate disodium, 0.2% sodium metabisulfite, 0.3% sodium thiosulfate, sodium hydroxide, and, adjusting the solution to about pH 5.5.

### Tetracycline Stabilizing Formulations

In certain embodiments, the present invention provides formulations for increasing the stability of tetracycline and its derivatives and analogs in aqueous solution. Formulations according to the invention comprise one or more chelating agents and one or more anti-oxidant agents. The pH and storage temperature of this formulation may also be adjusted.

The present invention provides stable tetracycline formulations, comprising a tetracycline, an anti-oxidant agent, a chelating agent and, if necessary, a pH adjusting agent in aqueous solution.

Chelating agents useful in the present invention may be any readily available, pharmaceutically acceptable chelating agent known in the art. For example, chelating agents which may be used in the invention include, without limitation, disodium edetate, ethylenediamine tetraacetic acid (EDTA), dimercaprol, diethylenetriaminepentaacetic acid (DTPA), N (hydroxyethyl) ethylenediaminetriacetic acid (HEDTA) and nitrilotriacetic acid (NTA). Chelating agents based on or derived from any of the above agents may also be used in the invention.

In certain embodiments, edetate disodium is added to the tetracycline solution in a range from about 0.001% to about 3.0% (weight per volume or weight per weight), or from about 0.001% to about 0.5%, or from about 0.1 to about 3%, or from about 0.001% to about 0.01%, from about 0.01% to about 0.1%, from about 0.1% to about 0.15%, from about 0.15% to about 0.20%, from about 0.20% to about 0.25%, from about 0.25% to about 0.30%, from about 0.30% to about 0.50%, from about 0.50% to about 1.0%, from about 1.0% to about 2.0%, or from about 2.0% to greater than 3.0%, i.e., about 3.0% to about 10.0% or greater. In a specific embodiment, about 0.20% edetate disodium is added to the tetracycline solution as a chelating agent.

Anti-oxidant agents utilized in the present invention may be any readily available anti-oxidant agent known in the medical art and preferably is pharmaceutically acceptable. For example, anti-oxidant agents used in the invention include, without limitation: sodium metabisulfite, sodium thiosulfate, sodium bisulfite and thiourea. Anti-oxidant agents based on or derived from any of the agents may also be used in the invention. In certain embodiments, the antioxidant agent is a combination of sodium metabisulfite and sodium thiosulfate. Other antioxidant agents may be used, alone or in various combinations.

In certain embodiments, sodium metabisulfite is added to the tetracycline solution in a range from about 0.001% to about 3.0% (weight per volume or weight per weight), or from about 0.001% to about 0.5%, or from about 0.1 to about 3%, or from about 0.001% to about 0.01%, from about 0.01% to about 0.1%, from about 0.1% to about 0.15%, from about 0.15% to about 0.20%, from about 0.20% to about 0.25%, from about 0.25% to about 0.30%, from about 0.30% to about 0.50%, from about 0.50% to about 1.0%, from about 1.0% to about 2.0%, or from about 2.0% to greater than 3.0%, i.e., about 3.0% to about 10.0% or greater. In a specific embodiment, about 0.20% sodium metabisulfite is added to the tetracycline solution as an anti-oxidant agent.

In certain embodiments, sodium thiosulfate is added to the tetracycline solution in a range from about 0.001% to about 3.0% (weight per volume or weight per weight), or from about 0.001% to about 0.5%, or from about 0.1 to about 3%, or from about 0.001% to about 0.01%, from about 0.01% to about 0.1%, from about 0.1% to about 0.15%, from about 0.15% to about 0.20%, from about 0.20% to about 0.25%, from about 0.25% to about 0.30%, from about 0.30% to about 0.50%, from about 0.50% to about 1.0%, from about 1.0% to about 2.0%, or from about 2.0% to greater than 3.0%, i.e., about 3.0% to about 10.0% or greater. In a specific embodiment, about 0.30% sodium thiosulfate is added to the tetracycline solution as an anti-oxidant agent.

The pH of a formulation of the invention may be adjusted using standard techniques in the art. The pH of the formulation may be adjusted to between about 3.0 and about 12.0. In certain embodiments, the pH of the formulation may be between about 4.0 and 11.0, about 5.0 and 10.0, about 6.0 and 9.0, or about 7.0 and 8.0. In other embodiments, the pH is, or is adjusted to be, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5, about 12.0, about 12.5 or about 13.0. In certain embodiments, the pH of the formulated solution is, or is adjusted to be, in a range from 4.5 to 7.5, or from about 4.5 to about 5.0, from about 5.0 to about 5.2, from about 5.2 to about 5.3, from about 5.3 to about 5.4, from about 5.4 to about 5.5, from about 5.5 to about 5.6, from about 5.6 to about 5.7, from about 5.7 to about 5.8, from about 5.8 to about 6.0, from about 6.0 to about 6.5, from about 6.5 to about 7.0, or from about 7.0 to about 7.5. In a specific embodiment, the pH of the tetracycline solution is or is adjusted to be about 5.5.

In certain embodiments, a stabilized tetracycline formulation is stored at freezing, refrigerated or room temperature. The storage temperature is in a range from about −25° C. to room temperature, or from −25° C. to about 6° C., or from about 3° C. to about 20° C., or from −25° C. to about −5.0° C., from −5.0° C. to about 0° C., from about 0° C. to about 2.0° C., from about 2.0° C. to about 3.0° C., from about 3.0° C. to about 3.5° C., from about 3.5° C. to about 4.0° C., from about 4.0° C. to about 4.5° C., from about 4.5° C. to about 5.0° C. to about to about 5.5° C., from about 5.5° C. to about 6.0° C., from about 6.0° C. to about 6.5° C., from about 6.5° C. to about 7.0° C., from about 7.0° C. to about 10.0° C., from about 10.0° C. to about 15° C., from about 15° C. to about 20° C., from about 20° C. to room temperature. In a specific embodiment, the tetracycline solution is stored at about 5.0° C.

In certain embodiments, the tetracycline formulation comprises one or more buffering agents.

In certain embodiments, the formulation further comprises light-protection of the tetracycline solution (e.g., packaging the solution in actinic glass or opaque materials).

In certain embodiments, the formulation further comprises dissolved gas, such as nitrogen gas, added for the purpose of replacing dissolved oxygen gas.

In certain embodiments, the present invention provides methods for slowing and/or decreasing the overall level of degradation of tetracycline in aqueous solution. Such a method comprises admixing in the solution a chelating agent and an anti-oxidant. In certain embodiments, the method further comprises adjusting the pH of the solution. In certain embodiments, the method further comprises storing the solution at temperatures below room temperature. In certain embodiments, the method further comprises protecting the solution from light (e.g., by packaging the solution in actinic glass or opaque materials). In certain embodiments, the method further comprises sparging the solution with a gas such as nitrogen gas. In a specific embodiment, the method comprises admixing in the solution 0.2% (w/w) edetate disodium, 0.2% sodium metabisulfite, 0.3% sodium thiosulfate, sodium hydroxide and adjusting the solution to pH 5.5. In more specific embodiments, the method further comprises storing the solution at or below about 5.0° C.

In certain embodiments, a buffering agent used in the invention is sodium phosphate dibasic and citric acid.

A tonicity modifier may also be utilized in the present invention. Such tonicity modifiers may be any readily available, pharmaceutically acceptable tonicity modifier known in the medical art. For example, tonicity modifiers used in the invention include, without limitation, sugars, glycerin, and sodium chloride.

In certain embodiments, glycerin is added to the tetracycline solution in a range from about 0.01% to about 5.0% (weight per volume or weight per weight), or from about 0.01% to about 1.5%, or from about 1.0% to about 5%, or from about 1.2% to about 1.5%, or from about 0.01% to about 1.0%, from about 1.0% to about 1.1%, from about 1.1% to about 1.2%, from about 1.2% to about 1.3%, from about 1.3% to about 1.4%, from about 1.4% to about 1.5%, from about 1.5% to about 1.6%, from about 1.6% to about 1.8%, from about 1.8% to about 2.0%, from about 2.0% to about 3.0%, from about 3.0% to about 5.0%. In a specific embodiment, about 1.2% glycerin is used as a tonicity modifier. In another embodiment, about 1.44% glycerin is used as a tonicity modifier.

In certain embodiments, the formulation comprises one or more buffering agents. The buffering agents utilized in the present invention may be any readily available, pharmaceutically acceptable buffering agent known in the medical art. For example, buffering used in the invention include, without limitation, phosphate-citrate buffer, phosphate buffer and acetate buffer.

In certain embodiments, a buffering agent used in the invention is a phosphate-citrate buffer, a combination of sodium phosphate dibasic and citric acid. The molar ratio of the two compounds that is required depends on the desired pH, and is well known by those skilled in the art. In a specific embodiment, about 0.10% sodium phosphate dibasic, anhydrous, and about 0.05% citric acid, anhydrous, is added to the tetracycline solution.

In certain embodiments, the formulation comprises an inert gas, e.g., Nitrogen (N₂). The gas may be introduced into the formulation by, e.g., sparging or other art acceptable means.

In certain embodiments, a tetracycline epimer is added to the tetracycline solution in a range of concentration from about 0.0001% to about 10.0% or greater (weight per volume or weight per weight), or from about 0.0001% to about 0.005%, or from about 0.001% to about 2.0%, or from about 0.0001% to about 0.0005%, from about 0.0005% to about 0.001%, from about 0.001% to about 0.0025%, from about 0.0025% to about 0.005%, from about 0.005% to about 0.01%, from about 0.01% to about 0.05%, from about 0.05% to about 0.1%, from about 0.1% to about 0.5%, from about 0.5% to about 2.0%, or from about 2.0% to greater than 5.0%, i.e., about 5.0% to about 10.0% or greater. In a specific embodiment, the tetracycline is doxycycline and the tetracycline epimer is 4-epidoxycycline.

In certain specific embodiments, the formulation for the tetracycline solution comprises: disodium edetate 0.1-0.5% (chelating agent), sodium metabisulfite 0.1-0.5% (antioxidant), sodium thiosulfate 0.1-0.5% (antioxidant), sodium phosphate dibasic 0.05-0.2% (buffer component), citric acid from about 0.025-0.1% (buffer component), glycerin 0.5-2% (tonicity modifier), and has or is adjusted to a pH of 5.2-5.8. Such a formulation for 0.025% w/w doxycycline, for example, has significantly longer shelf-life than was expected, with no detectable degradation after 12 weeks at 5° C. (see Table 1).

The tetracycline utilized in the present invention may be any readily available, pharmaceutically acceptable tetracycline known in the medical art. Included in this group of tetracyclines are those such as chlortetracycline, which is marketed under the tradenames Acronize®, Aureocina®, Aureomycin®, Biomitsin®, Biomycin® and Chrysomykine®; demeclocycline marketed as Ledermycin®, Detravis®, Meciclin®, and Mexocine®; doxycyline marketed as Vibramycin®, Vibramycin®, Hyclace®, Liomycin®, Vibradox®, Panamycin®, Titradox®, Hydramycin® and Tecacin®; lymecycline which is marketed as Armyl®, Mucomycin®, Tetramyl® and Tetralysal®; methacycline which is marketed as Adriamicina®, Cyclobiotic®, Germicilclin®, Globociclina®, Megamycine®, Pindex® and Londomycin®; Optimycin®, Rondomycin®; minocycline which is marketed as Minocin®, Klinomycin® and Vectrin®; oxytetracycline which is marketed as Biostat®, Oxacycline®, Oxatets®, Oxydon®, Oxymycin®, Oxytan®, Oxytetracid®, Ryomycin®, Stezazin®, Tetraject®, Terramycin®, Tetramel®, Tetran®, Dendarcin® and Dendarcin®; rolitetracycline marketed as Bristacin®, Revering, Superciclin®, Syntetrex®, Syntetrin®, Synotodecin®, Tetraverin®, Transcycline®, Velacicline® and Velacycline®; and tetracycline marketed as Achromycin®, Ambramycin®, Cyclomycin®, Polycycline®, Tetrabon® and Tetracyn®. In a specific embodiment, the tetracycline compound is doxycycline.

Active salts of tetracycline which are formed through protonation of the dimethylamino group on carbon atom 4, exist as crystalline compounds and are very stable in water. However, these amphoteric antibiotics will crystallize out of aqueous solutions of their salts unless stabilized by an excess of acid. Water soluble salts may be obtained also from bases such as sodium or potassium hydroxides but are not stable in aqueous solution, they are also formed with divalent and polyvalent metals.

In certain embodiments, the compounds useful according to the present invention are tetracyclines that have been chemically modified so as to substantially reduce or eliminate antimicrobial properties and/or to increase their anti-inflammatory activity. Methods for reducing antimicrobial properties of a tetracycline are disclosed, e.g., in The Chemistry of the Tetracyclines, Ch. 6, Mitscher, Ed., at p. 211. As pointed out by Mitscher, modification of tetracycline at positions 1, 2, 3, 4, 10 and 12a can lead to loss of antimicrobial activity. Such chemically modified tetracyclines (CMTs) are included in certain embodiments of the present invention because they can be used without disturbing the normal microbial flora of the treated subject as would be expected to happen with extended exposure to antimicrobial tetracyclines. Not all CMTs are non-antimicrobial at all concentrations. However, CMTs (as well as tetracyclines and analogs) may be administered, when desired, at concentrations to optimize anti-inflammatory activities and to reduce or minimize unwanted anti-microbial activities.

CMTs are useful in patients who are unable to tolerate tetracyclines for extended periods of time. The intolerance to tetracyclines can manifest itself in gastrointestinal problems, e.g., epigastric pain, nausea, vomiting, and diarrhea, or other problems related to taking long-term oral antibiotics. CMTs (or locally applied tetracyclines) can have greater efficacy because of the higher concentrations that can be achieved at the disease site. Because of their lack of antimicrobial-bacterial effect and greater therapeutic activity, many CMTs can have fewer systemic or other side effects than tetracyclines whether administered e.g., by intraocular injection, orally or topically.

Preferred CMTs used according to the present invention include those lacking a dimethylamino side chain at position 4. For example, 4-dedimethylamino-tetracycline, 4-dedimethylamino-5-oxytetracycline, 4-dedimethylamino-7-chlortetracycline, 4-hydroxy-4-dedimethylaminotetracycline, 4-dedimethylamino-12a-deoxytetracycline, 4-dedimethylamino-11-hydroxy-12a-deoxytetracycline, 4-dedimethylamino-7-dimethylaminotetracycline, 6-dimethyl-6-deoxy-4-dedimethylaminotetracycline, 6-O-deoxy-5-hydroxy-4-dedimethylaminotetracycline, 11a-chlortetracycline, 12a-deoxytetracycline, and the 2-nitrilo analogs of tetracycline.

In certain embodiments tetracycline, tetracycline analogs or CMTs may be formulated, alone or in combination, in a range of concentration from about 0.001% to about 20.0% or greater (weight per volume or weight per weight), or from about 0.001% to about to about 0.05%, or from about 0.01% to about to about 3%, from about 0.001% to about 0.01%, from about 0.01% to about 0.025%, from about 0.025% to about 0.05%, from about 0.05% to about 0.1%, from about 0.1% to about 0.25%, from about 0.25% to about 1.0%, from about 1.0% to about 2.0%, or from about 2.0% to greater than 3.0%, i.e., about 3.0% to about 10.0% or greater i.e., about 10.0% to about 20.0% or greater. In more specific embodiments, the concentration of tetracycline, tetracycline analog or CMT is from about 0.025% to about 0.05%.

### Additional Agents

In certain embodiments, the compositions of the invention may also contain adjuvants, such as preservatives, wetting agents, emulsifying agents, film-forming agents, and dispersing agents. Prevention of the action of microorganisms may be ensured by the inclusion of various antibacterial and antifungal agents, for example, paraben, chlorobutanol, phenol, sorbic acid, and the like.

In certain embodiments, the methods and compositions of the present invention comprise tetracycline, a tetracycline analog or CMT compound or composition as the sole therapeutic agent(s). In certain other embodiments, the present invention provides methods and compositions in which one or more of the subject compounds (e.g., tetracycline, tetracycline analogs or CMTs) is administered to a patient in need thereof in combination with one or more additional therapeutic agents. Additional therapeutic agents that may be useful in the compositions and methods of the present invention include, without limitation: anti-inflammatory agents (e.g., steroids such as, for example, triamcinolone acetonide or TA, which has been used experimentally in AMD, CME and DME, corticosteroids, glucocorticoids, macrolide antibiotics and the like), non-steroidal anti-inflammatory agents (NSAIDs) (e.g., carprofen, flurbiprofen, ibuprofen, niflumic acid, meclofenamic acid, ketoprofen, suxibutazone, diclofenac, mefenamic acid, tolfenamic acid, phenylbutazone and its metabolite oxyphenbutazone); metalloproteinase inhibitors (such as inhibitors to MMP-1, -2, -3, -7, -9, -13 and -14 which are present in eye tissues), immunosuppressive agents, anti-coagulants (e.g., low molecular weight heparin and various factors designed to promote blood coagulation), anti-angiogenic factors (e.g., various VEGF pathway inhibitors), retinoic acid derivatives (e.g., 9-cis-retinoic acid, 13-trans-retinoic acid and all-trans retinoic acid) vitamin D and its derivatives, estrogens, androgens, kinase inhibitors, growth factors, cytokines, vitamins and/or anti-oxidants.

In certain embodiments of the invention, the additional therapeutic agent is an anti-inflammatory agent. Exemplary anti-inflammatory agents that may be used in conjunction with the methods and compositions of the invention include, without limitation: dexamethasone, prednisone, prednisolone, betamethasone, budesonide, cortisone, hydrocortisone, methylprednisolone, prednisone and triamcinolone, cyclosporine, tacrolimus, pimecrolimus, loteprednol, fluoromethalone, rimexolone, ketorolac, diclofenac, bromfenac and nepafenac. Other exemplary anti-inflammatory mediators or agents that may be used in conjunction with the methods and compositions of the invention include cytokines known to work antagonistically to a host of inflammatory mediators and pro-inflammatory cytokines known in the art including, without limitation, IL-1, IL-6, IL-12/23p40, CXCLi2, IFN-gamma, IL-20 and TNF-alpha and their cognate receptors. Anti-inflammatory mediators include, without limitation: TGF-beta 1, TGF-beta 4, prostaglandin E(2), and various known prostaglandin inhibitors, such as, for example, flurbiprofen, as well as other cyclooxygenase-2 inhibitors such as, for example, celecoxib, indomethacin, meloxicam, nabumetone, nimesulide and rofecoxib.

In certain embodiments, the dosage of agents administered in combination with a tetracycline, an tetracycline analog, or CMT according to the present invention is dependent upon the age and weight of the patient being treated, the mode of administration, interactions between one or more compounds included in the composition (i.e., inhibitory, additive or synergistic) and the type and severity of the inflammatory or matrix-degrading disease being treated. Such factors are readily understood by the skilled practitioner.

In certain embodiments, the present invention provides compositions of stabilized tetracyclines in aqueous solution, ranging in concentration from about 0.001% to about 20.0% or greater. The concentration of tetracyclines depend on its method of use. A typical concentration for parenteral solution for antimicrobial use of, for example, doxycycline, is 0.1 to 1 mg/mL (equivalent to 0.01-0.1% w/w). A typical dose is about 100-200 mg/day (100-200 mL, assuming 1 mg/mL solution), infused over 1-4 hours. Typically, lyophilized powder is reconstituted into a 10% w/w solution, then further diluted into the final concentration with physiological saline (FDA guidelines). A subgingival formulation of doxycycline administered topically in a 10% solution is typically used for the treatment of periodontitis. 0.025-0.050% solution is typically used in treating dry-eye disease.

### Therapeutic Uses

In certain embodiments, the present invention provides methods for using the above-mentioned compositions of stabilized tetracyclines in aqueous solution for the treatment of inflammatory conditions, for example, acne vulgaris, rosacea, bullous dermatoses, rheumatoid arthritis, granulomatous disease, livedo vasculitis, sterile corneal ulceration, and periodontitis.

The above-mentioned aqueous tetracycline compositions, useful according to the methods of the invention, may be administered topically, systemically, or locally, for example, by ophthalmic or intranasal solution, injection, instillation, topical application, oral rinsing, or oral ingestion, to name a few.

In certain embodiments, the present invention provides methods for using the above-mentioned compositions of stabilized tetracyclines in aqueous solution for treatment of a patient suffering from a condition associated with a retinal and/or choroidal disease or disorder of the eye involving endothelial cell dysfunction, especially endothelial cells of the vasculature.

In certain embodiments, the present invention also provides methods for using the above-mentioned compositions of stabilized tetracyclines in aqueous solution for treatment of a patient suffering from conditions commonly termed “dry eye disease”—ocular surface diseases that result in reduced tear function or disruption of a proper tear film, and further results in irritation, inflammation, and/or erosion of the ocular surface epithelium, that can be caused by a variety of factors, including: blepharitis (including staphylococcal, seborrheic, anterior, and posterior blepharitis), meibomian gland disease, lacrimal glad dysfunction, hormonal changes due to menopause, various autoimmune diseases (e.g. rheumatoid arthritis, lupus, Sjogren's syndrome, etc.), and other dysfunctions of the tear component glands or neural loop controlling the tearing reflex. These methods for treatment are described elsewhere; see e.g., Pflugfelder et al. (U.S. Patent Application Publication No. 2008/0214510), the disclosure of which is incorporated by reference herein in its entirety.

In certain embodiments, the amount of tetracycline, tetracycline analog, or CMT administered considered to be effective is an amount that significantly decreases or inhibits one or more of: interleukin-1α; synthesis and activation of interleukin-1β or matrix metalloproteinases (MMP's); or conversion of precursor interleukin-1β to mature interleukin-1β according to the particular administration method or methods selected by the skilled practitioner. The maximal dosage for humans is the highest dosage that does not cause clinically important side effects.

The dosage or amount of tetracycline, tetracycline analog, or CMT to be administered to a patient as a formulation of the invention depends on a variety of factors that will be understood by the skilled practitioner and which may be further determined empirically. The effective serum concentration of a tetracycline compound may be selected depending on the patient, mode of administration, treatment regimen and desired goal and outcome of the treatment. Certain tetracyclines, tetracycline analogs, or CMTs will have different biological properties depending on their final concentration when administered to a patient. Antimicrobial activities elicited by one concentration may be reduced or eliminated by dilution without compromising desired anti-inflammatory activities, for example.

In certain embodiments, an amount of tetracycline considered to be effective is an amount that significantly decreases or inhibits one or more of: interleukin-1α; synthesis and activation of interleukin-1β or matrix metalloproteases (MMP's); or conversion of precursor interleukin-1β to mature interleukin-1β in the eye, which can be measured in tears using methods described in the art. The maximal dosage for humans is the highest dosage that does not cause clinically important side effects.

### EXAMPLES

The invention now being generally described, it will be more readily understood by reference to the following examples, which are included merely for purposes of illustration of certain aspects and embodiments of the present invention, and are not intended to limit the invention.

### Example 1

### Stabilized Tetracycline Formulations

A multi-dimensional parametric test on doxycycline stability was conducted to study the effect of pH, storage temperature and the presence of various chemical agents.

The experiments established that the rate of degradation is highly dependent on temperature. For example, about 51% of doxcycline in aqueous solution at neutral pH degraded after 2 weeks at room temperature (RT). In contrast, the same preparation had only 6% degradation when stored at 5° C.

Acidic pH also stabilized doxycycline in solution. Degradation after 2 weeks at RT and neutral pH was 51%, while degradation at pH 5.3 was 28%.

Various antioxidants also helped stabilize doxycycline in solution. Compared to the 27% degradation of doxycycline stored at pH 5.3 and RT for 2 weeks, doxycycline stored under the same conditions but with the addition of sodium metabisulfite, sodium thiosulfate, or thiourea had a degradation percentage of 8%, 5% and 20%, respectively. Not every antioxidant improved stability. Under the same conditions, the addition of sodium bisulfate did not appear to improve stability (28% degradation). For the full set of results, see Table 1.

TABLE 1
Multi-dimensional parametric testing of doxycycline stability
in aqueous solution.

| Antioxidant/ | API Content | | Storage | | | |
|---|---|---|---|---|---|---|
| Chelating Agent | [μg/mL] | pH | [° C.] | t = 0 | 2 wks | 4 wks |
| None | 250 | 5.3 | 5 | 100% | 96% | 93% |
| | | | 25 | | 73% | 59% |
| | | | 40 | | 19% | n.t. |
| | | 7 | 5 | 100% | 94% | n.t. |
| | | | 25 | | 49% | n.t. |
| | | | 40 | | 11% | n.t. |
| Sodium Bisulfite | 250 | 5.3 | 5 | 100% | 88% | n.t. |
| | | | 25 | | 72% | n.t. |
| | | | 40 | | 57% | n.t. |
| | | 7 | 5 | 100% | 91% | n.t. |
| | | | 25 | | 83% | n.t. |
| | | | 40 | | 48% | n.t. |
| Sodium | 250 | 5.3 | 5 | 100% | 99% | 97% |
| Metabisulfite | | | 25 | | 92% | 90% |
| | | | 40 | | 59% | n.t. |
| | | 7 | 5 | 100% | 97% | n.t. |
| | | | 25 | | 86% | n.t. |
| | | | 40 | | 51% | n.t. |
| Sodium | 250 | 5.3 | 5 | 100% | 99% | 99% |
| Thiosulfate | | | 25 | | 95% | 93% |
| | | | 40 | | 67% | n.t. |
| | | 7 | 5 | 100% | 96% | n.t. |
| | | | 25 | | 91% | n.t. |
| | | | 40 | | 7% | n.t. |
| Thiourea | 250 | 5.3 | 5 | 100% | 98% | n.t. |
| | | | 25 | | 80% | n.t. |
| | | | 40 | | 22% | n.t. |
| | | 7 | 5 | 100% | 98% | n.t. |
| | | | 25 | | 74% | n.t. |
| | | | 40 | | 9% | n.t. |
| Disodium Edetate | 250 | 5.3 | 5 | 100% | 99% | 96% |
| | | | 25 | | 97% | 88% |
| | | | 40 | | 24% | n.t. |
| | | 7 | 5 | 100% | 96% | n.t. |
| | | | 25 | | 55% | n.t. |
| | | | 40 | | 4% | n.t. |

Chelating agents also improved stability. For example, compared to the 28% degradation of doxycycline stored at pH 5.3 and RT for 2 weeks, the addition of edetate disodium resulted in a degradation percentage of 3%.

Further parametric studies showed that the combination of two antioxidants, sodium metabisulfite and sodium thiosulfate, provided better stabilization than sodium metabisulfite alone, for both doxycycline concentration (Table 2) as well as pH (Table 3).

TABLE 2
Combinatorial effect of antioxidants and tonicity adjustment agents in doxycycline stability

| Viscosity | | | Tonicity | | | | | | |
|---|---|---|---|---|---|---|---|---|---|
| Builder | Stabilizers | % w/w | (mOsm/kg) | Storage | Init. | 2 Wks | 5 Wks | 8 Wks | 12 Wks |
| HPMC | Edetate Disodium | 0.2% | Glycerin | 5° C. | 103.2% | 103.0% | 101.2% | 100.6% | 100.4% |
| | Sodium Metabisulfite | 0.2% | (287) | 25° C. | — | 101.0% | 93.3% | 85.2% | 84.8% |
| | Sodium Thiosulfate | 0.3% | | 40° C. | — | 80.3% | 55.1% | — | — |
| | | | NaCl | 5° C. | 103.4% | 103.0% | — | — | — |
| | | | (330) | 25° C. | — | 100.6% | — | — | — |
| | | | | 40° C. | — | 79.9% | — | — | — |
| | Edetate Disodium | 0.1% | Glycerin | 5° C. | — | | 102.8% | 100.6% | 99.4% |
| | Sodium Metabisulfite | 0.1% | (167) | 25° C. | — | | 97.0% | 90.4% | 85.1% |
| | Sodium Thiosulfate | 0.2% | | 40° C. | — | | 58.2% | | — |
| | | | NaCl | 5° C. | — | | 103.6% | — | — |
| | | | (325) | 25° C. | — | | 96.6% | — | — |
| | | | | 40° C. | — | | 55.8% | — | — |
| | Edetate Disodium | 0.1% | Glycerin | 5° C. | — | | 94.6% | — | — |
| | Sodium Metabisulfite | 0.1% | (138) | 25° C. | — | | 84.9% | — | — |
| | | | | 40° C. | — | | 47.1% | — | — |
| | | | NaCl | 5° C. | — | | 90.6% | — | — |
| | | | (305) | 25° C. | — | | 81.8% | — | |
| | | | | 40° C. | — | | 46.5% | — | — |

TABLE 3
Combinatorial effect of antioxidants and tonicity adjustment agents in pH stability

| Viscosity | | | Tonicity | | | | | | |
|---|---|---|---|---|---|---|---|---|---|
| Builder | Stabilizers | % w/w | (mOsm/kg) | Storage | Init. | 2 Wks | 5 Wks | 8 Wks | 12 Wks |
| HPMC | Edetate Disodium | 0.2% | Glycerin | 5° C. | 5.55 | 5.55 | 5.55 | 5.50 | 5.45 |
| | Sodium Metabisulfite | 0.2% | (287) | 25° C. | 5.55 | 5.60 | 5.30 | 5.00 | 4.65 |
| | Sodium Thiosulfate | 0.3% | | 40° C. | 5.55 | 4.85 | 4.00 | — | — |
| | | | NaCl | 5° C. | 5.55 | 5.55 | 5.40 | 5.45 | — |
| | | | (330) | 25° C. | 5.55 | 5.50 | 5.10 | 4.65 | — |
| | | | | 40° C. | 5.55 | 4.55 | 3.90 | — | — |
| | Edetate Disodium | 0.1% | Glycerin | 5° C. | 5.55 | 5.55 | 5.40 | 5.35 | 5.45 |
| | Sodium Metabisulfite | 0.1% | (167) | 25° C. | 5.55 | 5.50 | 5.10 | 5.05 | 4.85 |
| | Sodium Thiosulfate | 0.2% | | 40° C. | 5.55 | 5.00 | 4.20 | — | — |
| | | | NaCl | 5° C. | 5.55 | 5.75 | 5.60 | 5.50 | — |
| | | | (325) | 25° C. | 5.55 | 5.65 | 5.10 | 4.55 | — |
| | | | | 40° C. | 5.55 | 4.50 | 3.70 | — | — |
| | Edetate Disodium | 0.1% | Glycerin | 5° C. | 5.55 | 5.45 | 5.05 | — | — |
| | Sodium Metabisulfite | 0.1% | (138) | 25° C. | 5.55 | 5.00 | 4.10 | — | — |
| | | | | 40° C. | 5.55 | 4.45 | 3.65 | — | — |
| | | | NaCl | 5° C. | 5.55 | 4.90 | 3.80 | — | — |
| | | | (305) | 25° C. | 5.55 | 4.30 | 3.50 | — | — |
| | | | | 40° C. | 5.55 | 3.90 | 3.30 | — | — |

In addition, the pH was more stable in formulations using glycerin as the tonicity adjuster than in formulations using sodium chloride (Table 3).

Doxycyline in aqueous solution starts out as a clear and colorless liquid, but degradation results in the browning of the solution. Improved stability of doxycycline correlated well to the maintenance of solution color (Table 4).

TABLE 4
Multi-dimensional parametric testing of solution color in doxycycline
aqueous solution. Color is indicated by cell color (white - clear; grey - light
brown; black - dark brown)

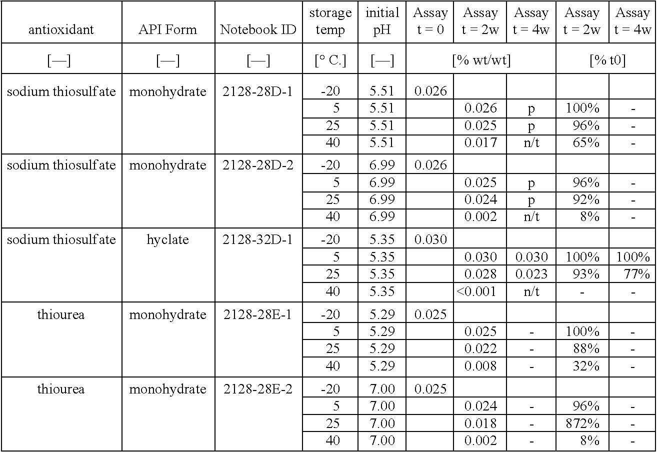

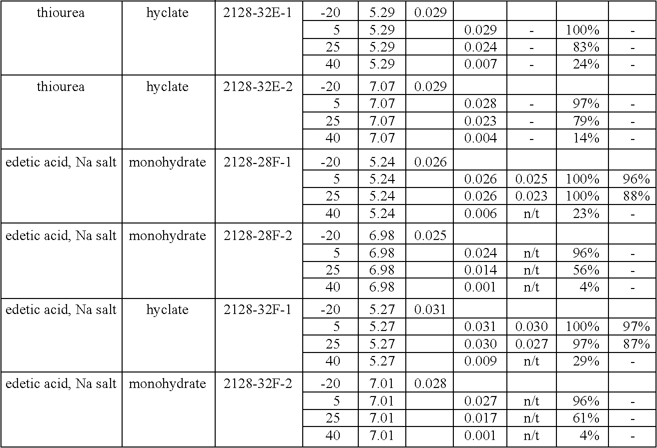

In a preferred formulation (see Table 8, phase 2 formulation), there were no changes in appearance (clear colorless without particulates), pH or doxycycline concentration at 5° C. storage conditions for a period of at least three months (see Table 2).

Further testing demonstrated that the pH of the phase 2 formulation remained stable, decreasing slightly from 5.5 to 5.2 after 12 months at 5° C. storage conditions, and the appearance remained clear colorless or pale yellow without particulates. The concentration of doxycycline stored at 5° C. in the preferred formulation was at approximately 95% of the initial value (% Label Claim) after 12 months (see Table 5). When the preferred formulation was stored at 25° C. (i.e., room temperature), the concentration of doxycycline was approximately 84% Label Claim after three months (see Table 5).

TABLE 5
Doxycycline formulation stability: 1-12 Months

| Buffer | | Storage | Doxycycline as % Label Claim | | | | | |
|---|---|---|---|---|---|---|---|---|
| Sr. | Process | Temp | Init. | 1 M | 2 M | 3 M | 9 M | 12 M |
| 9.5 mM | N₂ | 5° C. | 101.2% | 102.6% | 98.6% | 98.6% | 97.0% | 94.4% |
| | Sparged | 25° C. | | 96.6% | 87.9% | 83.4% | 69.4% | 63.4% |
| | Aerated | 5° C. | | 102.2% | 98.9% | 98.9% | 96.2% | 95.4% |
| | | 25° C. | | 97.2% | 88.2% | 83.8% | 68.0% | 63.6% |
| 0.45 mM | N₂ | 5° C. | 100% | 101.4% | 97.4% | 97.6% | 97.2% | 96.4% |
| | Sparged | 25° C. | | 97.0% | 87.7% | 83.6% | 67.6% | 62.2% |
| | Aerated | 5° C. | | 101.6% | 97.5% | 88.6% | 97.4% | 96.4% |
| | | 25° C. | | 97.6% | 85.9% | 84.3% | 67.6% | 61.0% |

### Example 2

### Epimerization and Degradation of Doxycycline

Doxycycline is known to undergo oxidative degradation and epimerization in aqueous solution (Libinson and Ushakova, Pharmaceutical Chemistry Journal, Vol. 10(8), pp. 91-93, 1976; Yekkala et al., Chromatographia, Vol. 31, pp. 313-316, 2003). Known potential impurities of doxycycline include, but are not limited to, 4-epidoxycycline, 6-epidoxycycline, 4,6-epidoxycycline, theacycline, and 2-acetyl-2-decarbixamidodoxycycline.

The rate of doxycycline epimerization increased with temperature and time (see Table 7). After 12 months of storage at 5° C., 0.025% w/w doxycycline formulation resulted in the appearance 0.0019% w/w 4-epidoxycycline. The same storage time at 25° C. resulted in the appearance of about 3 times as much 4-epidoxycycline (0.0060 w/w). Long-term storage (more than 18 months) should therefore be at refrigeration temperatures (2-8° C.) to minimize conversion of doxycycline to its epimers.

The determination of the rate of doxycycline epimerization also showed that epimerization is a major route of doxycycline degradation in aqueous solution. After 12 months of storage at 5° C., about 0.0014% w/w of the doxycycline in solution was lost to degradation. That is, original doxycyline concentration was 0.025% w/w and concentration after 12 months was 0.0236% w/w, corresponding to 94.4% Label Claim (LC). When the concentrations of particular epidoxycycline species were measured, the sample under 12-month storage was found to contain about 0.0019% w/w 4-epidoxycycline and about 0.0001% w/w 6-epidoxycycline (corresponding to 7.6% LC 4-epidoxcycline and 0.4% LC 6-epidoxycycline; see Table 7). Therefore, practically all of the degradation of doxycycline at 5° C. was through conversion into 4-epidoxycycline. Even at 25° C., where a larger portion of doxycycline degradation occurred through oxidation, epimerization into 4-epidoxycycline still accounted for over half of the degradation.

TABLE 7
Doxycycline epimerization at RT and under refrigeration

| 0.025% w/w | | | | | |
|---|---|---|---|---|---|
| Doxycycline in | Determined | % Label Claim | Change in | Determined | Determined |
| Standard | Doxycycline | for | Doxycycline % | 4-Epidoxycycline | 6-Epidoxycycline |
| Formulation | % w/w | Doxycycline | w/w | % w/w | % w/w |
| 5° C. for 12 mo | 0.0236 | 94.4 | −0.0014 | 0.0019 (7.6% LC) | 0.0001 (0.4% LC) |
| 25° C. for 12 mo | 0.0158 | 63.2 | −0.0092 | 0.0060 (24% LC) | 0.0001 (0.4% LC) |

The epimerization of doxycycline into 4-epidoxycycline is reversible, and the rate of 4-epidoxycycline formation is related to the kinetic equilibrium between the two compounds. Therefore, one method for specifically reducing the rate of doxycycline epimerization into 4-epidoxycycline is to fortify the formulation with 4-epidoxycycline. The addition of 0.00375% w/w 4-epidoxycycline into the 0.025% w/w doxycycline formulation results in reduced degradation of doxycycline during storage.

### Example 3

### Doxycycline Stability in Phase 3 Formulation

In further stability testing, the formulation was changed slightly. The first change was increasing the concentration of glycerin from 1.2 to 1.44%. This change brings the osmolality of the formula to a clinically more desirable level of about 270 mOsm/Kg. During stability evaluations it was also noted that compositions containing glycerin exhibit better pH stability.

The second change was increasing the level of sodium phosphate dibasic from 0.10% to 0.11% w/w. This was done to minimize the need to adjust the pH during the compounding of the product. The pH of laboratory batches made with the revised composition was found to be within the narrow pH specification. It should be noted that this is a minor change in the process where the entire amount of the buffer is added up front based on the experience gained during manufacturing. The final product pH and the buffer composition are still consistent with the previous composition used in phase 2.

Minor process improvements included: (i) sparging of the water with nitrogen prior to addition of the doxycycline monohydrate, (ii) reducing the water temperature to about 15° C. prior to the addition of the drug substance and (iii) heating the water to 50° C. prior to adding hydroxypropyl methylcellulose.

All of the above changes are minor and were incorporated to improve either the stability of API and components during processing or to make the product better suited for ophthalmic use and are not expected to have any adverse impact on either the product safety or efficacy. Table 8 compares the formula compositions of phase 2 and 3 materials.

TABLE 8
Composition of the phase 2 and phase 3 formulas

| | | % w/w | |
|---|---|---|---|
| | | Phase 2 | Phase 3 |
| Components | Function | Formula | Formula |
| Doxycycline | API | 0.0263 | 0.0263 |
| Monohydrate | | | |
| Edetate Disodium | chelating agent | 0.20 | 0.20 |
| Sodium | anti-oxidant | 0.20 | 0.20 |
| Metabisulfite | | | |
| Sodium Thiosulfate | anti-oxidant | 0.30 | 0.30 |
| Hydroxypropyl | viscosity | 0.35 | 0.35 |
| Methycellulose | modifier | | |
| Sodium Phosphate | buffering agent | 0.10 | 0.11 |
| Dibasic | | | |
| Citric Acid, | buffering agent | 0.05 | 0.05 |
| Anhydrous | | | |
| Glycerin | tonicity modifier | 1.20 | 1.44 |
| Sodium Hydroxide | pH adjustment | q.s. to pH 5.5 | q.s. to pH 5.5 |
| Hydrochloric Acid | pH adjustment | q.s. to pH 5.5 | q.s. to pH 5.5 |
| Purified Water | vehicle | q.s.a.d. | q.s.a.d. |

### Summary of Stability Test Results of Phase 3 Formula

Results from the doxycycline stability testing for the 5° C. samples are summarized in Table 9 for each of the strengths. Aside from the preferred doxycycline concentration of 0.025% w/w, lower concentrations of 0.0025 and 0.0050% w/w were also tested. The compositions exhibited acceptable chemical stability at this condition. The last column shows the slope of the linear regression plots; for the 0.025% strength, the rate of decrease in doxycycline content is 0.641% LC/month.

TABLE 9
Stability of doxycycline in phase 3 formulation at 5° C. storage

| Doxycycline | | | | | | | |
|---|---|---|---|---|---|---|---|
| Label Claim | Doxycycline Content (% LC) | | | | | | |
| (% ww) | T = 0 | 0.5 m | 1 m | 2 m | 3 m | 6 m | Slope* |
| 0.0025 | 97.5 | 96.0 | 97.3 | 98.7 | 96.6 | 94.6 | −0.394 |
| 0.0050 | 102.5 | 100.0 | 101.2 | 101.7 | 99.6 | 99.3 | −0.408 |
| 0.0250 | 102.9 | 101.6 | 102.0 | 102.1 | 101.7 | 98.4 | −0.641 |

*Slope of linear regression expressed as change in % doxycycline content/month

As has been found in earlier studies, the tetracycline in solution showed rapid degradation at 25° C. The results are shown in Table 10.

TABLE 10
Stability of doxycycline in phase 3 formulation at 25° C. storage

| Doxycycline | | | | | | |
|---|---|---|---|---|---|---|
| Label | | | | | | |
| Claim | Doxycycline Content (% LC) | | | | | |
| (% w/w) | T = 0 | 0.5 m | 1 m | 2 m | 3 m | 6 m |
| 0.0025 | 97.5 | 92.0 | 90.6 | 88.9 | 84.7 | 75.4 |
| 0.0050 | 102.5 | 98.0 | 95.0 | 91.5 | 86.1 | 77.6 |
| 0.0250 | 102.9 | 98.6 | 96.7 | 92.5 | 86.5 | 77.4 |

As seen from the data in Table 11, subjecting these compositions to three freeze-thaw cycles resulted in an approximately 3% loss of doxycycline. This decrease appears to be independent of the concentration of doxycycline in the product, and is most likely caused by exposure of the sample to 25° C. during each cycle. Because this product will be stored refrigerated over the long term, the freeze-thaw cycling studies may be repeated wherein each cycle consists of storing the product at −20° C. for three days followed by three to four days at 5° C. rather than storing at −20° C. for three days and 25° C. for three to four day as was done in this example.

TABLE 11
Effect of three freeze-thaw cycles on the doxycycline assay values

| | Doxycycline Assay | | |
|---|---|---|---|
| | Values (% Label Claim) | | |
| | | After three | |
| Label Claim | | freeze-thaw | % Loss of |
| (% w/w) | T = 0 | cycles | Doxycycline |
| 0.0025 | 97.5 | 94.8 | 2.8 |
| 0.0050 | 102.5 | 98.8 | 3.6 |
| 0.0250 | 102.9 | 99.8 | 3.0 |

Assay: 4 and 6-epidoxycycline

The formation of 4- and 6-epidoxycycline was assayed in 0.025% doxycycline in the phase 3 formulation. As has been observed in prior studies, the level of 6-epimer over 6 months at 5° C. did not show a significant increase with time. The level of 4-epimer, however, did increase with time of storage and reached a value of 3.99% LC at 6 months. The appearance stayed unchanged; they were all clear solutions. The pH remained essentially constant at 5° C.; over six months, the pH ranged from 5.5 to 5.3, while at T=0, it was 5.5. At 25° C., it ranged from 5.5 to 4.7 over the 6 months. Osmolality of the solutions was stable at around 280 mOsm.

TABLE 12
Stability of 0.05% doxycycline in phase 3 formula base

| Storage | | Time | Doxycycline | | | |
|---|---|---|---|---|---|---|
| Temp. | Batch # | (months) | (% LC) | 4-epimer | 6-epimer | pH |
| 5° C. | 2266- | Initial | 105.2 | 0.6 | 0.4 | 5.4 |
| | 122 | 1 | 102.4 | 0.6 | 0.3 | 5.5 |
| | | 3 | 104.3 | 2.3 | 0.4 | 5.5 |
| 25° C. | 2266- | Initial | 105.2 | 0.6 | 0.4 | 5.4 |
| | 122 | 1 | 97.2 | 3.4 | 0.3 | 5.3 |
| | | 3 | 89.2 | 13.4 | 0.4 | 4.8 |

Doxycycline solutions (0.05%) in the phase 3 base formulation were subjected to stability testing. Samples in glass vials were evaluated after one and three months at 5 and 25° C. The results were in keeping with the trends observed previously. The appearance stayed the same; the pH at 5° C. did not change; at 25° C., it dropped to previously observed values. The composition showed good chemical stability at 5° C., but at 25° C., the doxycycline content decreased with a concomitant increase in the degradation products. For example, the concentration of doxycycline was approximately 89% Label Claim after three months (see Table 12).

### Example 4

### Lack of Effectiveness of 4- and 6-Epidoxycycline in Treating Dry-Eye Disease

Previous studies report that 4-epidoxycycline does not function as an antibiotic. The lack of anti-inflammatory and anti-proteolytic effects of 4-epidoxycycline (as well as 6-epidoxycycline) was demonstrated in a treatment study for an experimental dry eye (EDE) model. In the study, EDE was induced in mice by subcutaneous scopolamine injection followed by exposure to an air draft and low humidity. This procedure typically causes desquamation (shedding) of endothelial cells in the apical cornea. These mice were grouped into the following treatment groups:

| | |
|---|---|
| DOXY | Doxycycline 0.025% |
| DOXY 10 | Doxycycline 0.0025% |
| EPI6 | 6-epidoxycyline 0.025% |
| EPI4 | 4-epidoxycyline 0.025% |
| DOXY + EPI4 | Doxycycline 0.0125% and 4-epidoxycyline 0.0125% |
| DOXY + EPI6 | Doxycycline 0.0125% and 6-epidoxycyline 0.0125% |
| VEHICLE | Dow formulation vehicle |
| 5 D | 5 days of Experimental dry eye |
| | (w/o therapeutic treatment) |
| UT | untreated control (no EDE, no therapeutic treatment) |

Subjects in each group (4 animals each) were treated during the course of EDE with the corresponding formulation. Following EDE and treatment, whole-mount preparations of the corneas were immuno-stained for occluding (a tight-junction protein) to distinguish individual epithelial cells in the apical cornea and observed under laser-scanning confocal microscopy to detect desquamation.

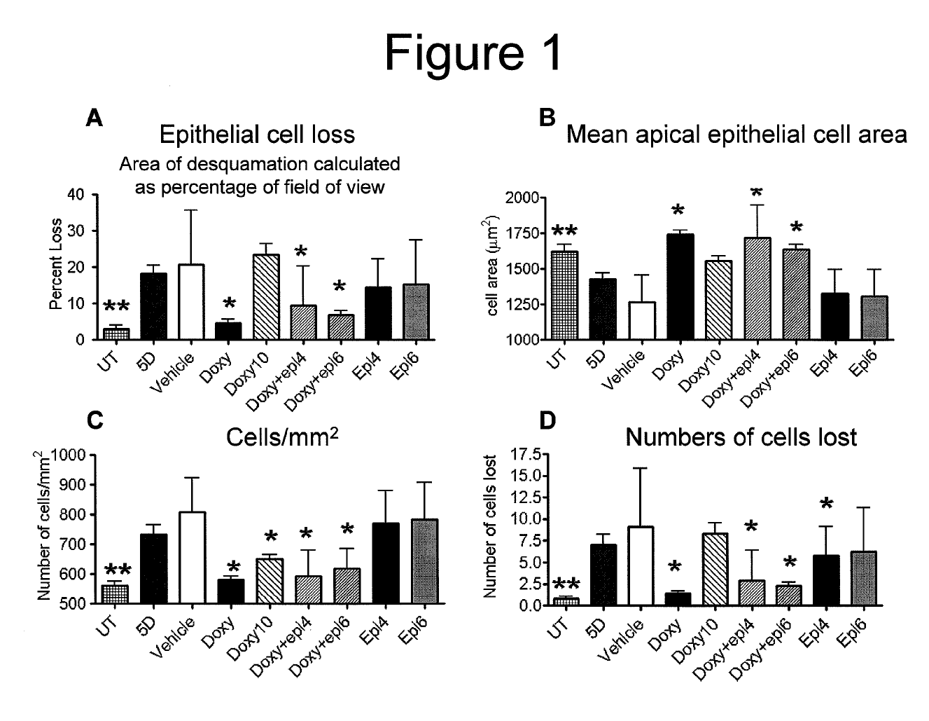

Five days of EDE resulted in cell desquamation from almost 20% of the apical surface of the cornea, while the level of desquamation in untreated controls (not exposed to EDE) was <5% (See FIG. 1A). Treatment with 0.025% doxycycline resulted in the level of desquamation being significantly lower compared to vehicle treatment, and similar to the level measured in the untreated control group, demonstrating the effectiveness of 0.025% doxycycline in preventing corneal damage due to dry eye disease (see FIG. 1). However, a ten-fold lower dose of 0.0025% doxycyline was ineffective in preventing corneal damage due to EDE, and resulted in a level of desquamation similar to vehicle treatment (although even this concentration showed some effectiveness when desquamation was measured by cell density; see FIG. 1C).

This study also demonstrated that the major epimers of doxycycline (4- and 6-epidoxycycline) were ineffective and failed to prevent corneal damage due to dry eye. Treatment with 0.025% 4-epidoxycycline or 0.025% 6-epidoxycycline during EDE resulted in levels of desquamation similar to, and in most measures statistically indistinguishable from, treatment with vehicle (see FIG. 1). These results further demonstrate that 4-epidoxycycline and 6-epidoxycycline lack the anti-inflammatory and anti-proteolytic properties of doxycycline, which is at least one mechanism thought to account for the compound's ability to protect the cornea from damage due to dry eye.

The DOXY+EPI4 and DOXY+EPI6 formulations (doxycycline 0.0125% with 4-epidoxycyline 0.0125%, and doxycycline 0.0125% with 4-epidoxycyline 0.0125%, respectively) approximate doxycycline treatment where 50% of the doxycycline in a 0.025% solution has degraded due to epimerization. These two formulations were still effective (in varying measures depending on how desquamation was measured) in reducing desquamation due to dry eye (see FIG. 1).

What is claimed is:

1. A tetracycline composition comprising a tetracycline formulated in an aqueous solution comprising a chelating agent at a concentration of about 0.1-0.5%, and an antioxidant agent at a concentration of about 0.1-0.5%, wherein the pH of the solution is between about 4.5 and about 7.5.

2. The composition of claim 1, wherein the chelating agent is disodium edetate.

3. The composition of claim 1, wherein the antioxidant agent is selected from the group consisting of: sodium bisulfate, sodium metabisulfite, sodium thiosulfate, and thiourea.

4. The composition of claim 3, wherein the antioxidant agent is a combination of sodium metabisulfite and sodium thiosulfate.

5. The composition of claim 1, further comprising a buffering agent.

6. The composition of claim 5, wherein the buffering agent comprises sodium phosphate dibasic and citric acid, wherein the concentration of sodium phosphate dibasic is from about 0.05% to about 0.2% and the concentration of citric acid is from about 0.025% to about 0.1%.

7. The composition of claim 1, further comprising a tonicity modifier.

8. The composition of claim 7, wherein the tonicity modifier is glycerin, wherein the concentration of glycerin is from about 0.5% to about 2%.

9. The composition of claim 1, wherein the pH of the aqueous solution is between about 5.2 and about 5.8.

10. The composition of claim 1, wherein the tetracycline is in the monohydrate or hyclate form.

11. The composition of claim 1, wherein the tetracycline is a tetracycline analog.

12. The composition of claim 11, wherein the tetracycline analog is selected from the group consisting of: oxytetracycline, doxycycline and minocycline.

13. The composition of claim 12, wherein the tetracycline analog is doxycycline, wherein the concentration of the doxycycline is from about 0.0025% to about 1%.

14. The composition of claim 1, wherein the tetracycline is a chemically modified tetracycline.

15. A pharmaceutical composition comprising a tetracycline formulated in an aqueous solution comprising a chelating agent at a concentration of about 0.1-0.5%, an antioxidant agent at a concentration of about 0.1-0.5%, and a pharmaceutically acceptable carrier, wherein the pH of the solution is between about 4.5 and about 7.5.

16. The composition of claim 15, wherein the chelating agent is disodium edetate.

17. The composition of claim 15, wherein the antioxidant agent is selected from the group consisting of: sodium bisulfite, sodium metabisulfite, sodium thiosulfate, and thiourea.

18. The composition of claim 17, wherein the antioxidant agent is a combination of sodium metabisulfite and sodium thiosulfate.

19. The composition of claim 15, further comprising a buffering agent.

20. The composition of claim 19, wherein the buffering agent comprises sodium phosphate dibasic and citric acid, wherein the concentration of sodium phosphate dibasic is from about 0.05% to about 0.2% and the concentration of citric acid is from about 0.025% to about 0.1%.

21. The composition of claim 15, further comprising a tonicity modifier.

22. The composition of claim 21, wherein the tonicity modifier is glycerin, wherein the concentration of glycerin is from about 0.5% to about 2%.

23. The composition of claim 15, wherein the pH of the aqueous solution is between about 5.2 and about 5.8.

24. The composition of claim 15, wherein the tetracycline is in the monohydrate or hyclate form.

25. The composition of claim 15, wherein the tetracycline is a tetracycline analog.

26. The composition of claim 25, wherein the tetracycline analog is selected from the group consisting of: oxytetracycline, doxycycline and minocycline.

27. The composition of claim 26, wherein the tetracycline analog is doxycycline, wherein the concentration of the doxycycline is from about 0.0025 to about 1%.

28. The composition of claim 15, wherein the tetracycline is a chemically modified tetracycline.

29. A method for decreasing the degradation of a tetracycline in aqueous solution, comprising admixing in the solution a chelating agent at a concentration of about 0.1-0.5% and an antioxidant at a concentration of about 0.1-0.5%, and, adjusting the pH so that the pH in the solution is between about 4.5 and about 7.5.

30. The method of claim 29, wherein the chelating agent is disodium edetate.

31. The method of claim 29, wherein the antioxidant is selected from the group consisting of: sodium bisulfite, sodium metabisulfite, sodium thiosulfate, and thiourea.

32. The method of claim 31, wherein the antioxidant agent is a combination of sodium metabisulfite and sodium thiosulfate.

33. The method of claim 29, further comprising a step of admixing in the solution a buffering agent.

34. The method of claim 33, wherein the buffering agent comprises sodium phosphate dibasic and citric acid, wherein the concentration of sodium phosphate dibasic is from about 0.05% to about 0.2% and the concentration of citric acid is from about 0.025% to about 0.1% citric acid.

35. The method of claim 29, further comprising a step of admixing in the solution a tonicity modifier.

36. The method of claim 35, wherein the tonicity modifier is glycerin, wherein the concentration of glycerin is from about 0.5% to about 2%.

37. The method of claim 29, wherein the pH of the aqueous solution is between about 5.2 and about 5.8.

38. The method of claim 29, wherein the tetracycline is in the monohydrate or hyclate form.

39. The method of claim 29, wherein the tetracycline is a tetracycline analog.

40. The method of claim 39, wherein the tetracycline analog is selected from the group consisting of: oxytetracycline, doxycycline and minocycline.

41. The method of claim 40, wherein the tetracycline analog is doxycycline, wherein the concentration of doxycycline is from about 0.0025% to about 1%.

42. The method of claim 29, wherein the tetracycline is a chemically modified tetracycline.

43. A method for treating a patient suffering from a condition associated with inflammation and/or tissue degeneration, said method comprising administering to a patient in need thereof a composition comprising an effective amount of a tetracycline in an aqueous solution, further comprising a chelating agent at a concentration of about 0.1-0.5%, and an antioxidant agent at a concentration of about 0.1-0.5%, wherein the pH of the solution is between about 4.5 and about 7.5.

44. The method of claim 43, wherein the chelating agent is disodium edetate.

45. The method of claim 43, wherein the antioxidant agent is selected from the group consisting of: sodium bisulfate, sodium metabisulfite, sodium thiosulfate, and thiourea.

46. The method of claim 45, wherein the antioxidant agent is a combination of sodium metabisulfite and sodium thiosulfate.

47. The composition of claim 43, further comprising a buffering agent.

48. The composition of claim 47, wherein the buffering agent is a phosphate citrate buffer wherein the concentration of sodium phosphate dibasic is from about 0.05% to about 0.2% and the concentration of citric acid is from about 0.025% to about 0.1%.

49. The composition of claim 43, further comprising a tonicity modifier.

50. The composition of claim 49, wherein the tonicity modifier is glycerin, wherein the concentration of glycerin is from about 0.5% to about 2%.

51. The method of claim 43, wherein the pH of the aqueous solution is between about 5.2 and about 5.8.

52. The method of claim 43, wherein the tetracycline is in the monohydrate or hyclate form.

53. The method of claim 43, wherein the tetracycline is a tetracycline analog.

54. The method of claim 53, wherein the tetracycline analog is selected from the group consisting of: oxytetracycline, doxycycline and minocycline.

55. The method of claim 54, wherein the tetracycline analog is doxycycline, wherein the concentration of the doxycycline is from about 0.0025% to about 1%.

56. The method of claim 43, wherein the tetracycline is a chemically modified tetracycline.

57. The method of claim 43, wherein said condition associated with inflammation or tissue degeneration is selected from the group consisting of: acne vulgaris, rosacea, bullous dermatoses, rheumatoid arthritis, granulomatous disease, livedo vasculitis, sterile corneal ulceration, dry eye disease, macular degeneration, recurrent corneal corrosion, and diabetic retinopathy.

58. The method of claim 43, wherein said condition associated with inflammation or tissue degeneration is dry eye disease.

59. An aqueous solution comprising a chelating agent at a concentration of about 0.1-0.5%, and an antioxidant agent at a concentration of about 0.1-0.5%, wherein the pH of the solution is between about 4.5 and about 7.5.

60. The composition of claim 59, wherein the chelating agent is disodium edetate.

61. The composition of claim 59, wherein the antioxidant agent is selected from the group consisting of: sodium bisulfite, sodium metabisulfite, sodium thiosulfate, and thiourea.

62. The composition of claim 61, wherein the antioxidant agent is a combination of sodium metabisulfite and sodium thiosulfate.

63. The composition of claim 59, further comprising a buffering agent.

64. The composition of claim 63, wherein the buffering agent comprises sodium phosphate dibasic and citric acid, wherein the concentration of sodium phosphate dibasic is from about 0.05% to about 0.2% and the concentration of citric acid is from about 0.025% to about 0.1%.

65. The composition of claim 59, further comprising a tonicity modifier.

66. The composition of claim 65, wherein the tonicity modifier is glycerin, wherein the concentration of glycerin is from about 0.5% to about 2%.

67. The composition of claim 59, wherein the pH of the aqueous solution is between about 5.2 and about 5.8.
